(19) United States
(12) Patent Application Publication
(10) Pub. No.: US 2022/0096258 A1
(43) Pub. Date: Mar. 31, 2022

(54) LATCH WIRE AND DRIVER SHAFT

(71) Applicant: Boston Scientific Scimed, Inc., Maple Grove, MN (US)

(72) Inventor: James M. Anderson, Corcoran, MN (US)

(21) Appl. No.: 17/477,978

(22) Filed: Sep. 17, 2021

Related U.S. Application Data
(60) Provisional application No. 63/083,315, filed on Sep. 25, 2020.

(51) Int. Cl. A61F 2/966; A61F 2/24; A61F 2/95

(52) U.S. Cl. CPC A61F 2/966; A61F 2/2466; A61F 2002/9505; A61F 2002/9665; A61F 2/9517

(57) ABSTRACT

Improvements to devices and systems for delivering and/or deploying an implantable device. The device includes at least one latch extending from a flexible elongate member to engage with the implantable device, such as during delivery and/or deployment. The flexible elongate member includes a locking region on the exterior thereof and shaped and configured to be distinct from surrounding areas. The locking region may be engaged with one or more of the latch, a component (e.g., a control knob) of a control handle of the system, or a driver shaft/actuator extending along (e.g., over) the flexible elongate member to drive or actuate a component of the implantable device, such as to move, implant, adjust, etc. the implantable device.

### PRIORITY

The present application is a non-provisional of, and claims the benefit of priority under 35 U.S.C. § 119 to, U.S. Provisional Application Ser. No. 63/083,315, filed Sep. 25, 2020, the disclosure of which is hereby incorporated herein by reference in its entirety for all purposes.

### FIELD

The present disclosure relates generally to improvements to medical devices, systems, and methods for use with, such as for delivery and/or deployment of, implantable medical devices.

### BACKGROUND

Various implantable medical devices are implanted with the use of a delivery/deployment device or system which delivers and implants the device at a treatment site. Some such implantable devices are implanted within the body to an extent requiring a flexible elongate member of the delivery/deployment system to maneuver to the treatment site along a tortuous path.

One challenge presented by such delivery/deployment devices and systems is that the flexible elongate member and latch wires are twisted through the tortuous path to the treatment site, and then may be twisted further if used to rotate an element or component of the implantable device to implant the implantable device. Additionally, aligning latch wires into latch housings during assembly may be very tedious and time consuming. Set screws used to couple the latch wires with other components of the devices or systems may not have a secure hold on typical wires with round cross-sections, and additional manners of securing, such as adhesives, may be needed. Moreover, the set screws may cause torque on the latch wire when tightened, and may interfere with proper latch alignment, which may cause difficulties during use, loading, and/or deployment. And, set screws may strip out in plastic knobs used in control handles for maneuvering the device to the treatment site.

It is with considerations of these and other challenges in mind that improvements such as disclosed herein may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a delivery and deployment system for delivering and/or deploying an implantable device is provided with a control handle configured to manipulate components of the delivery and deployment device, a flexible elongate member having a proximal end coupled to the control handle and a distal end extending distally therefrom, and a latch coupled to the distal end of the flexible elongate member. In one aspect, the flexible elongate member includes a first locking region, formed on an exterior thereof, shaped and configured to engage with one of the control handle or the latch to inhibit relative movement therebetween; and the one of the control handle or the latch includes a channel for receiving an end of the flexible elongate member with a channel locking region corresponding to the first locking region for engagement therewith to inhibit relative movement between the flexible elongate member and the one of the control handle and the latch.

In some embodiments, the first locking region has a shape and/or configuration distinct from the contour of the flexible elongate member surrounding the first locking region. In some embodiments, the first locking region is at least one of a flat, a swaged region, or an enlarged region.

In some embodiments, the system further includes at least one latch control knob associated with the control handle, the proximal end of the flexible elongate member engaging the control handle via the latch control knob. The latch control knob defines a channel for receiving the proximal end of the flexible elongate member and having a locking region corresponding to and engaging with the first locking region to inhibit relative movement between the flexible elongate member and the latch control knob.

In some embodiments, the first locking region is a flat on the exterior of the flexible elongate member, and the control handle includes a press-fit locking element extending through a portion of the control handle and into engagement with the flat.

In some embodiments, the system further includes a driver shaft defining a channel therethrough, the flexible elongate member extending through the channel in the driver shaft. In some embodiments, the driver shaft channel includes a driver shaft channel locking region, and the flexible elongate member includes a second locking region formed in an exterior thereof shaped and configured to engage with the driver shaft channel locking region to inhibit relative rotation, while allowing axial movement, between the flexible elongate member and the driver shaft. In addition or alternatively, the control handle may include at least one actuator control knob; and the driver shaft may have a proximal end coupled to the actuator control knob and a distal end coupled to a driver coupler configured to engage a component of an implantable device, movement of the actuator control knob controlling movement of the driver coupler via the driver shaft. In addition or alternatively, the control handle may include at least one latch control knob located proximal to the actuator control knob, the flexible elongate member extending through the actuator control knob and into the latch control knob. In some embodiments, the first locking region and the second locking region are separate. In some embodiments, the second locking region is an extension of the first locking region.

In accordance with various principles of the present disclosure, an implantable device delivery/deployment system is provided with a flexible elongate member having a proximal end coupled to the control handle and a distal end extending distally therefrom, a latch coupled to the distal end of the flexible elongate member, an implantable device having a coupler corresponding to and configured to be engaged with the latch, and a control handle configured to manipulate components of the delivery/deployment system to deliver and deploy the implantable device. The flexible elongate member includes a first locking region formed in an exterior thereof shaped and configured to engage with one of the control handle or the latch to inhibit relative movement therebetween.

In some embodiments, the one of the control handle or the latch includes a channel for receiving an end of the flexible elongate member with a channel locking region corresponding to the first locking region for engagement therewith to inhibit relative movement between the flexible elongate member and the one of the control handle and the latch.

In some embodiments, the implantable device delivery/deployment system further including a driver shaft defining a channel therethrough and having a proximal end coupled to the control handle and a distal end extending distally therefrom configured to engage a component of the implantable device to deploy the implantable device at a treatment site. The flexible elongate member extends through the channel defined through the driver shaft. The driver shaft includes a driver shaft channel locking region, and the flexible elongate member includes a second locking region formed in an exterior thereof shaped and configured to engage with the driver shaft channel locking region.

In some embodiments, the implantable device delivery/deployment system further includes a latch control knob coupled to the proximal end of the flexible elongate member, a driver coupler coupled to the distal end of the driver shaft and configured to extend over the latch and corresponding coupler on the implantable device, and an actuator control knob coupled to the proximal end of the driver shaft. Actuation of the actuator control knob actuates the driver shaft to rotate to manipulate a portion of the implantable device, or to move axially relative to the latch to sheathe or unsheathe the latch and the corresponding coupler on the implantable device. Rotational movement between the flexible elongate member and the driver shaft is inhibited by engagement of the second locking region and the driver shaft channel locking region.

In accordance with further principles of the present disclosure, a method of delivering and deploying an implantable device to a treatment site is disclosed, the method including coupling a latch at a distal end of a flexible elongate member extending from a delivery/deployment system to a coupler on the implantable device, sheathing the coupled latch and coupler with a driver shaft extending from the delivery/deployment system to maintain engagement between the latch and the coupler during delivery of the implantable device to the treatment site, and navigating the flexible elongate member and driver shaft to deliver the implantable device to the treatment site. The flexible elongate member extends through a channel defined in the driver shaft and has a locking region on an exterior thereof engaged with a corresponding locking region in the driver shaft channel to inhibit relative rotational movement between the flexible elongate member and the driver shaft.

In some embodiments, the method further includes actuating the driver shaft via an actuator control knob on a control handle of the delivery/deployment system to implant an anchor of the implantable device into the treatment site.

In some embodiments, the method further includes actuating the driver shaft via an actuator control knob to adjust at least one of the size, shape, configuration, or dimension of the implantable device.

In some embodiments, the method further includes extending a driver coupler on a distal end of the driver shaft over the latch and engaging the driver coupler with the coupler on the implantable device, and rotating the driver shaft via an actuator control knob to rotate a component of the implantable device coupled to rotate with the coupler to adjust at least one of the size, shape, configuration, dimension, or position of the implantable device, the flexible elongate member engaging within and reinforcing rotation of the driver shaft.

In some embodiments, the method further includes moving the driver shaft proximally with respect to the flexible elongate member to allow the latch to disengage from the corresponding coupler on the implantable device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is a perspective view of an example of a delivery/deployment system formed in accordance with aspects of the present disclosure with an example of an implantable device in a collapsed configuration.

FIG. 2 is a perspective view of an example of a control handle assembly of a delivery/deployment system in accordance with aspects of the present disclosure.

FIG. 3 is a cross-sectional view of an example of a control knob assembly of a delivery/deployment system in accordance with aspects of the present disclosure.

FIG. 4 is a cross-sectional view through a latch control knob partially exploded to show a locking element in accordance with aspects of the present disclosure.

FIG. 5 is a perspective view of an alternate latch wire in accordance with aspects of the present disclosure.

FIG. 6 is an end view of a latch knob in accordance with aspects of the present disclosure.

FIG. 7 is an end view of an alternate latch knob in accordance with aspects of the present disclosure.

FIG. 8 is a cross-sectional view of a latch in accordance with aspects of the present disclosure.

FIG. 9 is a partial perspective view of a distal end of a latch wire configured for placement within a latch as in FIG. 8.

FIG. 10 a partial perspective view of a distal end of a latch wire configured for placement within a latch as in FIG. 8.

FIG. 11 is a perspective view of a latch knob, driver knob, latch, and driver shaft isolated from a delivery/deployment system in accordance with aspects of the present disclosure.

FIG. 12 is an end perspective view of a driver shaft in accordance with aspects of the present disclosure.

FIG. 13 is an end perspective view of an alternative driver shaft in accordance with aspects of the present disclosure.

FIG. 14 is a perspective view of a delivery/deployment system formed in accordance with aspects of the present disclosure and shown delivering an implantable device in a schematic representation of an example of a treatment site.

FIG. 15 is a perspective view of an example of an implantable device configured for delivery at an implantation site by a delivery/deployment system as in FIG. 1.

FIG. 16 is a partial perspective view of a distal portion of a delivery/deployment system implanting and adjusting an implantable device in accordance with aspects of the present disclosure

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably without intent to limit or otherwise), etc., such as when using a device (e.g., introducing the device into a patient, or during delivery, deployment, implantation, positioning, etc.), and “distal” refers to the direction or location furthest from the user, such as when using the device (e.g., introducing the device into a patient, or during delivery, deployment, implantation, positioning, etc.). “Longitudinal” means extending along the longer or larger dimension of an element. “Central” means at least generally bisecting a center point, and a “central axis” means, with respect to an opening, a line that at least generally bisects a center point of the opening, extending longitudinally along the length of the opening when the opening comprises, for example, a tubular element, a strut, a channel, or a bore.

In accordance with one aspect of the present disclosure, a system for deploying and/or delivering an implantable device (referenced herein as a delivery/deployment device for the sake of simplicity and without intent to limit), as disclosed herein with reference to various embodiments, includes a delivery/deployment device structure with at least one flexible elongate member (such as in the form of a wire, shaft, etc.) capable of being navigated through a tortuous path within the human body and configured to engage a portion of the implantable device (e.g., to deliver the implantable device to a treatment site, to deploy the implantable device, to implant the implantable device, to manipulate the implantable device, etc.). In accordance with various aspects of the present disclosure, the flexible elongate member has an improved configuration which may reduce torque generated during delivery and/or deployment and/or implantation and/or adjustment (such terms being used interchangeably herein with one another or other similar terms without intent to limit) of the implantable device. In some embodiments, a latch or coupler or connector (such terms being used interchangeably herein without intent to limit, reference generally being made to a latch for the sake of convenience and without intent to limit) is positioned at a distal end of the flexible elongate member to engage the implantable device, such as during delivery, deployment, implantation, etc. of the device. The latch may be shaped and configured to engage a corresponding structure, such as a latch or coupler or connector (such terms being used interchangeably herein without intent to limit, reference generally being made to a coupler for the sake of convenience, such as to distinguish from the latch of the delivery/deployment system and without intent to limit) on the implantable device. The latch on the delivery/deployment system may facilitate coupling or engagement of the implantable device with the delivery/deployment system during delivery of the implantable system. The flexible elongate member may extend proximally to be controlled by a control handle, such as by a latch control knob.

In some embodiments, an actuator or driver or driver shaft or driver tube (such terms being used interchangeably herein without intent to limit), separate from the latch and flexible elongate member, is provided to manipulate the implantable device (e.g., to actuate a component of the implantable device to facilitate implantation thereof, or to manipulate the implantable device at the treatment site, such as to adjust the position, configuration, etc., of the implantable device). The actuator may include an elongated driver component, such as a driver shaft, extending between a controller (e.g., a knob) at a proximal end of the elongated driver component, and a driver coupler at a distal end of the elongated driver component configured to engage the implantable device to actuate a component thereof (e.g., impart movement thereto). The actuator may interact with, such as by being engaged or coupled with, the flexible elongate member and driver coupler and latch.

In accordance with one aspect of the present disclosure, the flexible elongate member has an exterior contour with at least one locking region having a shape and/or configuration distinct from the contour of the exterior portion of the flexible elongate member surrounding the locking region. The locking region may be at a distal end of the flexible elongate member, a proximal end of the flexible elongate member, both ends of the flexible elongate member, and/or along the length of the flexible elongate member between the proximal end and the distal end.

A locking region at a distal end of the flexible elongate member may facilitate coupling of a latch thereto, such as during assembly, and may improve the connection therebetween. Engagement of a latch with the locking region inhibits movement (e.g., rotation, which may potentially weaken the connection between the flexible elongate member and the latch) of the flexible elongate member relative to the latch. Reduction or elimination of relative movement between the delivery/deployment system latch (at the end of the flexible elongate member) and the flexible elongate member may result in corresponding reduction or elimination of movement of the latch relative to an implantable device engaged with or coupled to (such as to be carried via) the latch, such as upon navigation of the flexible elongate member and the implantable device to the treatment site, or upon manipulation of the flexible elongate member to manipulate the implantable device. For instance, if the component of the implantable device coupled with the latch is rotated to implant or adjust the implantable device, engagement of the delivery/deployment system latch with a locking region on the flexible elongate member inhibits or prevents potential relative movement between the delivery/deployment system latch and the flexible elongate member during such rotation.

A locking region at a proximal end of the flexible elongate member may facilitate coupling of the flexible elongate member to a controller or control knob to inhibit relative movement, such as rotation, between the control knob and the flexible elongate member. Such movement may occur as a result of active movement (e.g., rotation) of the control knob, such as to move or maneuver the flexible elongate member and latch, or transferred movement resulting from movement of the flexible elongate member and/or latch distal to the control knob (e.g., during navigation through a tortuous path, or as a result of other movement of an implantable device coupled to a distal end of the flexible elongate member and/or latch). Alternatively or additionally, if an additional actuator or driver or driver shaft is provided to manipulate the implantable device and is associated or coupled with the flexible elongate member and/or the delivery/deployment system latch, engagement of the control knob with a locking region on the flexible elongate member inhibits or prevents potential relative movement the flexible elongate member may transfer proximally to the control knob as a result of movement of the flexible elongate member caused by actuation (and movement) of the actuator. A locking region on the flexible elongate member may also permit use of improved locking mechanisms for coupling a control knob to the proximal end of the flexible elongate member. In embodiments in which the flexible elongate member extends through an actuator or driver shaft, a distal end of the flexible elongate member may extend proximally beyond the proximal end of the driver shaft, the proximal end of the flexible elongate member being coupled to a latch control knob and the proximal end of the driver shaft being coupled to an actuator control knob, the latch control knob being proximal to the actuator control knob. In some embodiments, the driver shaft is axially movable with respect to and over the flexible elongate member, such as by moving the actuator control knob relative to the latch control knob. The distal end of the flexible elongate member may extend distally beyond the distal end of the driver shaft to couple the latch (on the distal end of the flexible elongate member) with a coupler on the implantable device, and the driver shaft may be selectively movable axially to sheathe or unsheathe the latch and coupler.

In some embodiments, the flexible elongate member may include a locking region interacting or engageable with a component of the actuator or driver. The actuator may be used during implantation of the implantable device (such as to actuate a component of the implantable device to be implanted into the treatment site) and/or to engage or carry or deliver or otherwise the implantable device to the treatment site. The actuator (and optionally its associated driver coupler) and the flexible elongate member and the latch may assist with maintaining connection between the delivery/deployment device and the implantable device. A locking region on the flexible elongate member engaging the actuator may improve various operations and/or functions of the actuator. For instance, engagement of locking regions on the flexible elongate member and the actuator may enhance torque on the actuator such as for implanting the implantable device.

In some embodiments, an implantable device is provided with a latch or coupler or connector corresponding to the delivery/deployment system latch and coupled thereto during delivery and/or deployment and/or implanting of the implantable device by the delivery/deployment system. The implantable device may be delivered in a low profile (e.g., retracted or compressed or otherwise smaller configuration than when the device is deployed so that the device may be maneuvered through the body to the treatment site). In some embodiments, the implantable device may be delivered to a treatment site within a delivery sheath. At least one latch on a delivery/deployment device structure of the delivery/deployment system may be coupled with a corresponding coupler on the implantable device during delivery to a treatment site. Alternatively or additionally, at least one latch on a delivery/deployment device structure of the delivery/deployment system may be coupled with a corresponding coupler on the implantable device to manipulate the implantable device at the treatment site, such as to adjust one or more aspects of the implantable device, such as implantation (in the treatment site), position (overall, or of a component of the delivery/deployment device), configuration, etc.

In some embodiments, the delivery/deployment system is used to implant an implantable device in the form of a generally tubular frame having a proximal end and a distal end. In one embodiment, the frame may be configured for custom reshaping of a heart valve, such as the mitral valve. The frame may include, at at least one end thereof, one or more anchors that may be translatably advanced into tissue at a treatment site. One or more anchor housings may be provided, each carrying or housing an anchor.

According to one aspect of the disclosure, a flexible elongate member may include a latch configured to engage with a latch or coupler on a proximal end of at least one anchor on an implantable device. An actuator or driver shaft may be used to advance or drive the anchor into the treatment site (e.g., valve annulus), and/or to adjust one or more of the anchors of the implantable device once anchored in the desired location. The actuator or driver shaft may be selectively movable over the latch and coupler and may rotate both to adjust the position of the anchor relative to the treatment site.

Alternatively or additionally, it may be desirable to adjust the overall implant size and/or shape to adjust the size and/or shape of the valve in which the implantable device has been implanted. In some embodiments, the implantable device may be configured (such as with struts joined along distal and proximal apices) to be expandable or retractable (e.g., radially) to move or otherwise be adjusted between a reduced-diameter configuration for delivery and an expanded-diameter configuration for implantation. It may be desirable to further adjust the overall diameter or configuration of the implantable device once implanted, in order to adjust the shape of the valve, such as to effect improved closure thereof. Cinch devices may be provided on the implantable device and movable to adjust the relative positions of the anchors (generally, once the anchors have been implanted) to adjust the configuration of the implantable device and/or the implant location. For instance, in some embodiments the implantable device is implanted around a cardiac valve, such as the mitral valve or the tricuspid valve, with a plurality of spaced apart anchors. Adjustment of the relative positions of one or more anchors once implanted around the valve adjusts the shape of the valve to facilitate repair of the valve. In some embodiments, a cinch collar or collar or slider (such terms being used interchangeably herein without intent to limit) may be provided over components of the implantable device to draw together such components or to allow such components to move apart to affect the shape and configuration of the implantable device and, consequently, the shape and configuration of the tissue to which the implantable device is secured. For instance, in an implantable device formed with struts joined at distal and proximal apices, the collar may be positioned over the proximal apices and advanced or retracted to affect the relative orientations of adjacent struts to affect the configuration of the frame. An actuator or driver shaft (such as that used to implant the anchors) may be engaged with the implantable device, such as with the collar or an actuator thereof (e.g., a rotatable shaft advancing or retracting the collar upon being rotated by the actuator), to adjust the implantable device. The flexible elongate member and associated latch may be coupled to a coupler on the collar or the collar actuator. The actuator or driver shaft may be engaged over the latch and the coupler and rotate both to adjust the position of the collar to adjust the implantable device.

Various embodiments of a deployment/delivery device and system and associated method will now be described with reference to examples illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present invention is not limited to only the embodiments specifically described herein.

Turning now to the drawings, it will be appreciated that in the following description, elements or components similar among the various illustrated embodiments are generally designated with the same reference numbers and redundant description is omitted. Common features are identified by common reference elements (generally differing in value by a factor of 100), and, for the sake of brevity, the descriptions of the common features are generally not repeated.

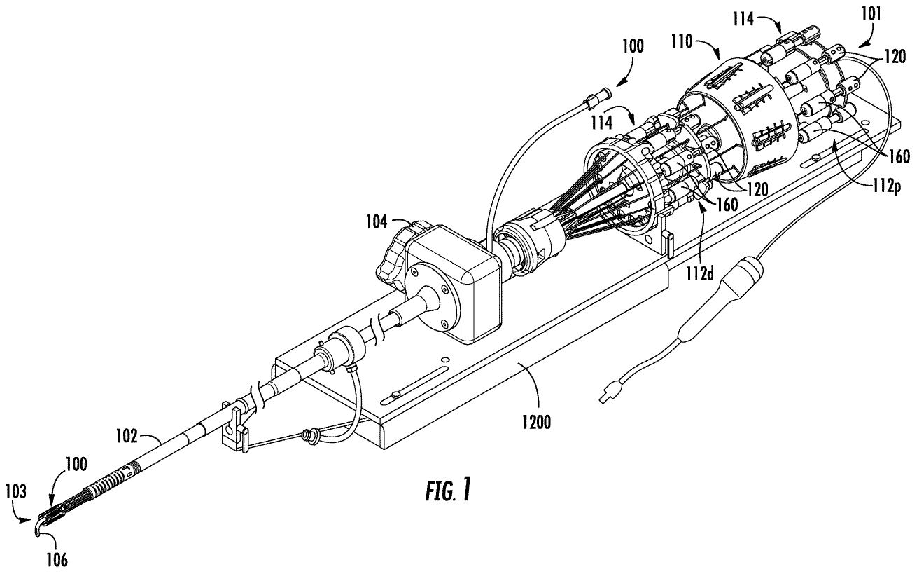

An example of a system 100 for delivery and/or deployment of (or other action associated with) an implantable device 1000 (carried by or coupled to or otherwise associated with the delivery/deployment system 100 and which may be considered in some instances as part of the delivery/deployment system 100) to a treatment site is shown, in a perspective view, in FIG. 1. The system 100 will be referenced herein as a delivery/deployment system 100 to convey the optional multi-use aspect of the system without intent to limit the system to a single or particular use in connection with an implantable device 1000. Moreover, references to delivery/deployment systems are to be understood as optionally including an implantable device. The delivery/deployment system 100 may include a steerable delivery device 102 (e.g., catheter, sheath, or the like) through which the implantable device 1000 may be delivered (e.g., transluminally), and which may be controlled (e.g., steered or navigated) by a delivery device control knob 104.

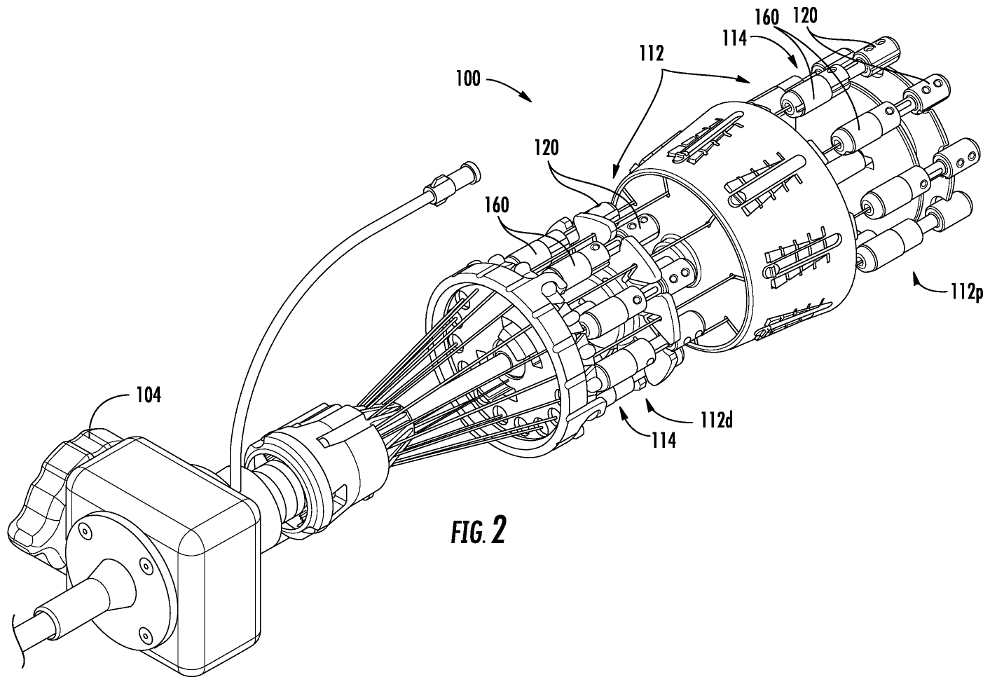

The delivery/deployment system 100 may include one or more devices for imaging capabilities, such as an imaging catheter 106, such as an ultrasound catheter or intravascular cardiac echography (ICE) catheter. An example of a steerable delivery device and system with various positioning and imaging capabilities is described in U.S. Pat. No. 10,335,275, titled METHODS FOR DEPLOYMENT OF HEART VALVE DEVICES USING INTRAVASCULAR ULTRASOUND IMAGING, and issued on Jul. 2, 2019, which patent is incorporated herein by reference in its entirety for all purposes. A control handle assembly 110, illustrated in greater detail in FIG. 2 (and which may be provided in a delivery/deployment system 100 as in FIG. 1), is provided at a proximal end 101 of the delivery/deployment system 100. A stage or stand 1200 may be provided to support the control handle assembly 110. The control handle assembly 110 includes one or more control sections 112, and may also include the delivery device control knob 104. Each control section 112 may include one or more knobs 114. Each knob may be configured for and capable of controlling (e.g., steering or operating) a different component of the delivery/deployment system 100. Different knobs 114 may be provided to effect or implement different operations or actions or control movements on the implantable device 1000. Although the control sections 112 are illustrated as a proximal control sections 112p and a distal control section 112d, other relative arrangements of the control sections 112 (such as peripherally with respect to one another) are within the scope of the present disclosure.

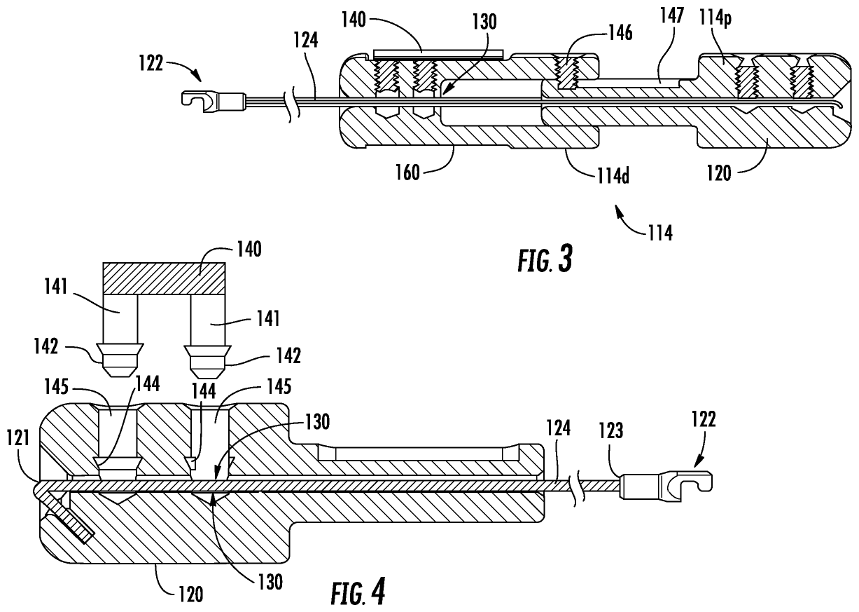

In the embodiments illustrated in FIGS. 1 and 2, each control section 112 has at least one control knob 114 configured and arranged for controlling at least one component of the delivery/deployment system 100 and/or at least one component at a distal end 103 of the delivery/deployment system 100. As illustrated in conjunction with FIGS. 3 and 4, at least one of the control knobs 114 includes a knob 120 coupled with a latch or coupler or connector 122 (such terms being used interchangeably herein without intent to limit, reference being made more commonly to a latch 122 merely for the sake of convenience) via a flexible elongate member 124 (e.g., a shaft or wire). Such knob will be referenced herein as a latch knob 120 for the sake of convenience without intent to limit to a particular term such as “latch.” The latch 122, on a distal end 123 of the flexible elongate member 124 (with the latch knob 120 on the proximal end 121 of the flexible elongate member 124), is configured to be engaged, coupled, or otherwise with a corresponding element (latch or coupler or connector or the like) on the implantable device 1000 (examples being described in further detail below). The latch knob 120 may be manipulated, moved (rotatably, axially, etc.), or otherwise to manipulate, move, etc. (such terms being used interchangeably herein without intent to limit) the latch 122 in or out of engagement with the corresponding element on the implantable device 1000, or to manipulate, move, etc. the implantable device 1000, as will be described below in further detail with reference to an example of an implantable device 1000.

In some embodiments, some of the knobs 114 are coupled with other components of the delivery/deployment system 100. For instance, a knob may be provided to actuate or control an actuator or driver or driver shaft (such terms being used interchangeably herein without intent to limit) coupled to the implantable device 1000 to manipulate or operate or actuate or control (such terms being used interchangeably herein without intent to limit) a component of the implantable device 1000, such as to implant the implantable device 1000. In some embodiments, as described in further detail below, one or more knobs may be provided to control one or more driver shafts 164 configured to transmit torque to a component of an implantable device 1000 (e.g., an anchor 1120 or a collar actuator 1136 of a collar 1130) to implant and/or adjust or otherwise manipulate or move the implantable device 1000.

In accordance with various principles of the present disclosure, various embodiments of the flexible elongate member 124 include at least one locking region 130 formed along an exterior thereof. In some embodiments, such locking region 130 has a shape and/or configuration distinct from the surrounding exterior contour of the flexible elongate member 124 to facilitate engagement or coupling of the flexible elongate member 124 with another component, such as a latch knob 120 or a latch 122 (e.g., for coupling or mounting of the latch knob 120 or the latch 122 with or on the flexible elongate member 124). The locking region 130 may be formed in various manners which modify the exterior of the flexible elongate member 124, such as, without limitation, stamping, swaging, grinding, machining, or otherwise shaping features into wire, or using profile drawn wires and shafts tubing.

Referring now to various embodiments illustrated in FIGS. 3-7, in accordance with one aspect of the present disclosure, a flexible elongate member 124 may be provided with at least one flat (e.g., a region of the flexible elongate member 124 which is substantially flat or has a radius of curvature significantly larger than that of surrounding regions) extending along part or all of the length of the flexible elongate member 124. As may be appreciated with reference to FIG. 3 and FIG. 4, the flexible elongate member 124 may be a substantially flat wire element (e.g., with a flat region on substantially opposite sides and extending along the length of the flexible elongate member 124), or have a flat region of limited longitudinal extent on each side, as illustrated in FIG. 5. Other extents and locations of a flat region are within the scope and spirit of the present disclosure. An embodiment of a locking region 130 formed of at least a portion of the flat region of such a flexible elongate member 124 presents a substantially flat region with respect to a locking element 140 used to hold the flexible elongate member 124 in place with respect to the latch knob 120. For example, if the locking element 140 is in the form of one or more set screws, such as illustrated in FIG. 3, the flexible elongate member 124 presents a substantially flat surface for engagement with an end of the set screw, providing a more secure engagement then would be provided by a typical wire with a round cross-section. In some embodiments, the locking element 140 may be in the form of a press-fit locking element having an extension or post or pin element 141 extended through channels within the latch knob 120 and into engagement with the proximal end 121 of the flexible elongate member 124, such as a push-pin lock as illustrated in FIG. 4. The push-pin locking element simply needs to be pushed into locking engagement with the locking region 130 of the flexible elongate member 124 without rotation, such as necessary with typically small set screws, thereby facilitating assembly of the control handle assembly 110. The push-pin lock may have locking ends 142 (e.g., ribs or grooves) configured to engage with corresponding locking features 144 (e.g., corresponding grooves or ribs) within locking bores 145 in the latch knob 120. Such press-fit locking element 140 typically would be faster and easier to assemble (in contrast with set screws), and can be readily disassembled and reassembled.

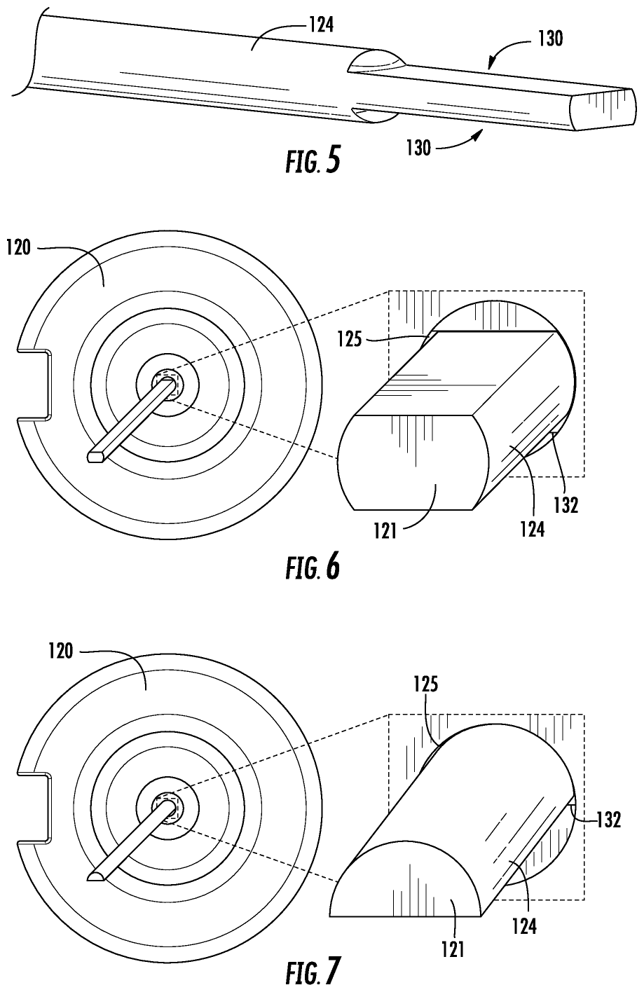

As may be appreciated with further reference to FIG. 6 and FIG. 7, the channel 125 in the knob 120 through which the proximal end 121 of the flexible elongate member 124 extends may have a locking region 132 corresponding (e.g., sized and/or shaped and/or configured and/or located with respect) to the locking region 130 on the flexible elongate member 124 for engagement or coupling therewith to inhibit or prevent relative movement (e.g., rotation or axial translation) of the flexible elongate member 124 within the latch knob 120, further enhancing secure coupling of the latch knob 120 and the flexible elongate member 124. More particularly, as may be appreciated with reference to the end views of the latch knob 120 illustrated in FIG. 6, such as in conjunction with FIGS. 3 and 4, the channel 125 may have a locking region 132 with flat inner wall regions corresponding to the flats of a locking region 130 of a flexible elongate member 124 as shown in FIGS. 3-5. Alternatively, if the locking region 130 has a single flat, then the channel 125 may have a corresponding locking region 132 with a corresponding single flat region, as illustrated in FIG. 7. Such corresponding configurations of a portion of the channel 125 and a portion of the exterior of the flexible elongate member 124 may facilitate alignment of these elements during assembly, and results in a more resilient reliable connection less dependent on additional components such as set screws or adhesive than prior art connections.

Similar concepts for locking regions may be applied to the distal end 123 of the flexible elongate member 124 for facilitating engagement or coupling with a latch 122, with corresponding locking regions provided within a channel within the latch receiving a distal end of the flexible elongate member 124, similar to as described above with respect to the latch knob 120. For the sake of brevity, reference is made to the above-described concepts applied to the connection of the flexible elongate member 124 and latch knob 120 as being similarly applicable to the connection of the flexible elongate member 124 and the latch 122, with accompanying similar benefits.

It will be appreciated that various alternative configurations of locking regions are within the scope of the present disclosure, such as other shapes which inhibit relative movement such as relative rotation.

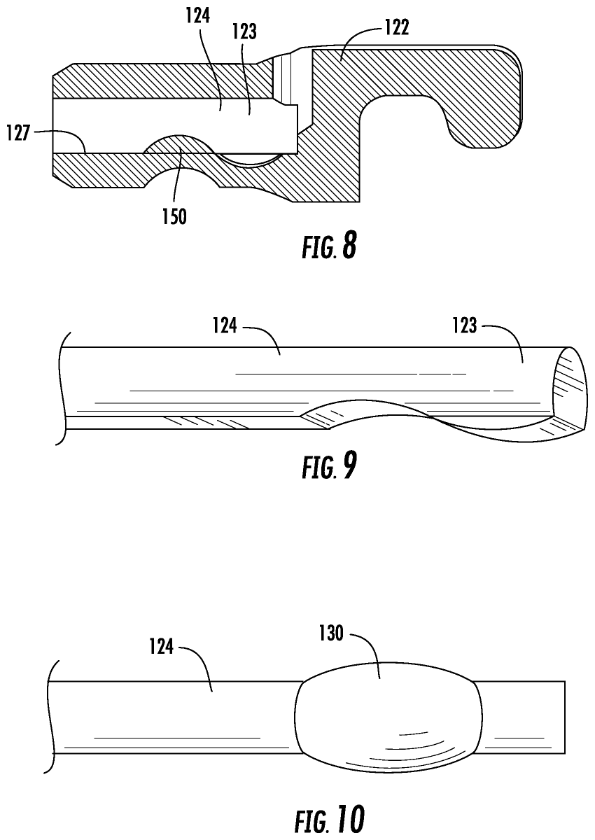

Alternatively or additionally, the locking region 130 of the flexible elongate member 124 may be formed by an alternate modification to a portion of the exterior of the flexible elongate member 124, such as to lock or otherwise be securely engaged or coupled within a latch knob 120 or latch 122 or the like. For instance, the proximal end 121 or distal end 123 of the flexible elongate member 124 may be press fit (e.g., cold pulled) or swaged or enlarged (e.g., by addition of material thereto) or formed or otherwise, examples of which are illustrated in FIGS. 8-10.

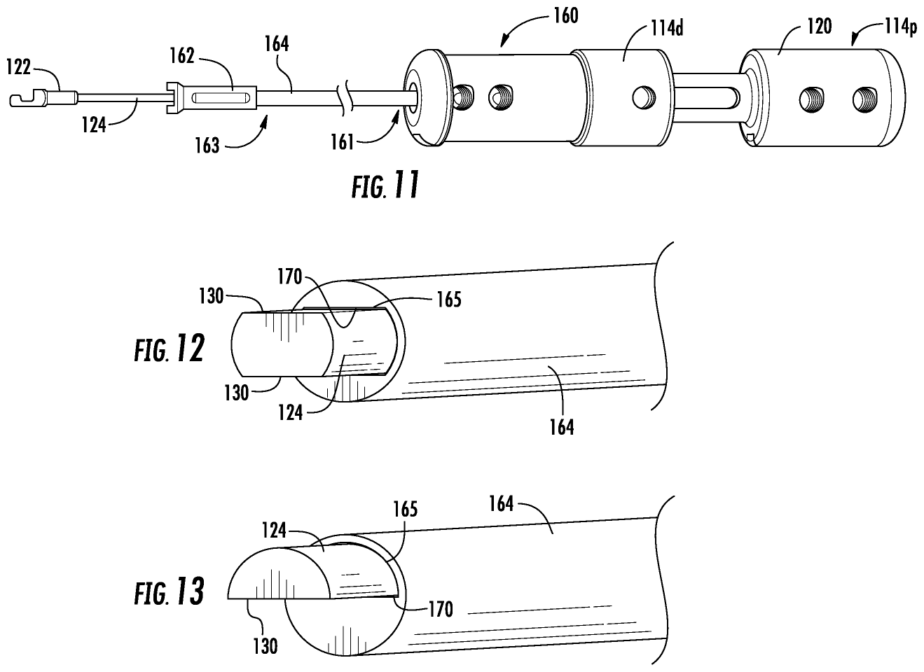

As illustrated in FIG. 8, in some embodiments a latch 122 may include a locking element 150 (such as a projection or groove, or material shaped, disposed, added, deformed, or manipulated or otherwise to a mechanical locking advantage) extending within the channel 127 of the latch 122 through which the distal end 123 of the flexible elongate member 124 extends. A distal end 123 of a flexible elongate member 124, as illustrated in FIG. 9 may be swaged to correspond in shape and location with the locking elements 150 in the latch. To further enhance the engagement, such as to secure against relative rotation of the latch 122 and flexible elongate member 124, the flexible elongate member 124 may also include a flat surface or region (a “flat”) engaging a flat within the channel 127 in the latch 122. Alternatively, the locking region 130 on the flexible elongate member 124 may be in the form of an enlarged region, as illustrated in FIG. 10. The locking element 150 in the latch 122 may be configured to correspond with the enlarged region to enhance coupling of the latch 122 and the flexible elongate member 124. For instance, the locking element 150 may be in the form of a groove or mechanically locking fitment or otherwise receiving the enlarged region on the flexible elongate member 124. Or, the enlarged region may simply pass over a locking element 150 in the form of a rib. It will be appreciated that an enlarged region, such as on the flexible elongate member 124, may be formed of a deformable material (e.g., Nitinol, such as in a superelastic state) which deforms to pass over another region and then recover or return to its prior shape to lock in place mechanically. If the circumferential extent of the enlarged region is not limited (e.g., extends around the perimeter of the flexible elongate member 124), and an additional flat may be provided to inhibit relative rotation between the latch 122 and the flexible elongate member 124.

Returning to FIGS. 1-3, in one aspect of the present disclosure, at least one of the control knobs 114 of the control handle assembly 110 is formed as a two component knob, comprising a proximal control knob 114p and a distal control knob 114d. In the illustrated embodiment, the proximal control knob 114p is the latch knob 120 and the distal control knob 114d is an actuator knob 160, although different arrangements are within the scope of the present disclosure. The proximal control knobs 114p and distal control knobs 114d may be coupled together, such as to be axially movable with respect to each other but to be inhibited from relative rotational movement. For instance, as illustrated in FIG. 3, a locking pin 146 may extend from one of the control knobs 114p, 114d, into a longitudinal groove or channel 147 in the other of the control knobs 114p, 114d. Other arrangements are within the scope of the present disclosure.

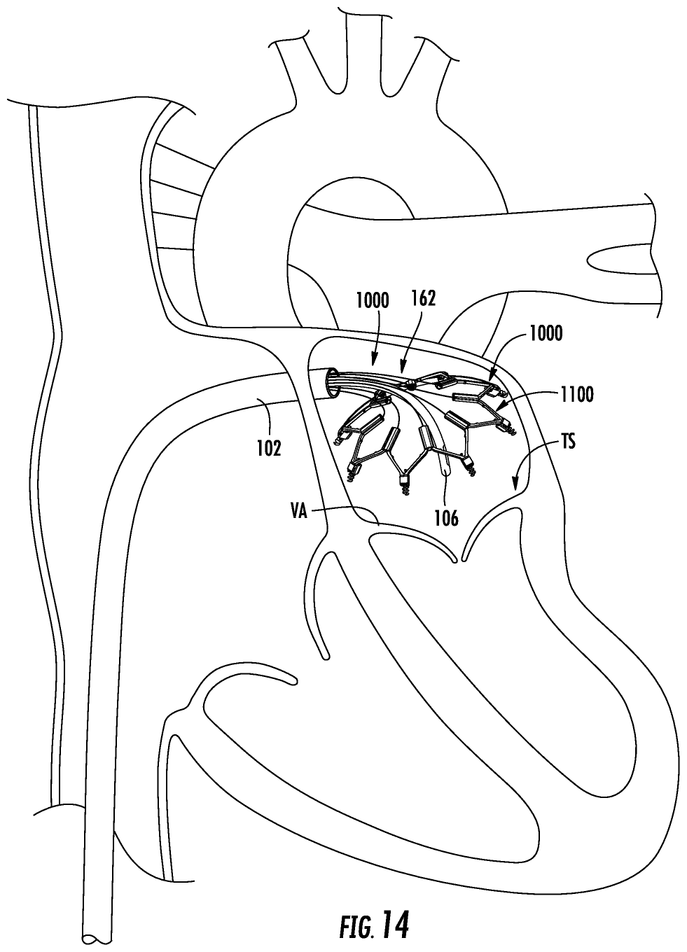

As illustrated in FIG. 11, the actuator knob 160 is coupled to an actuator or driver coupler or driver 162 (such terms being used interchangeably herein without intent to limit) via a driver shaft 164. The driver 162, on a distal end 163 of the driver shaft 164 (with the actuator knob 160 on the proximal end 161 of the driver shaft 164), is configured to be engaged or coupled with a component of the implantable device 1000 to manipulate or move or actuate (such terms being used interchangeably herein with one another and other such terms without intent to limit) the implantable device 1000, such as to actuate a component of the implantable device 1000 to facilitate implantation thereof, or to manipulate the implantable device 1000 at the treatment site, such as to adjust the position, configuration, etc., of the implantable device 1000. The actuator knob 160 may be manipulated, moved (rotatably, axially, etc.), or otherwise to manipulate, move, etc. (such terms being used interchangeably herein without intent to limit) the driver 162 to effect the desired action on the implantable device 1000, as will be described below in further detail with reference to an example of an implantable device 1000.

In the configuration of the embodiment illustrated in FIG. 11, the flexible elongate member 124 extends through a channel 165 within the driver shaft 164. The flexible elongate member 124 extends out through the proximal end 161 of the driver shaft 164 to be coupled with the latch knob 120, and out through the distal end 163 of the driver shaft 164 to engage or be coupled with an implantable device 1000 (such as via a latch 122). The latch knob 120 is movable (e.g., axially translatable) with respect to the actuator knob 160 (such as facilitated by a mating slot/screw arrangement between the latch knob 120 and the actuator knob 160). In some embodiments, as described in further detail below, the latch 122 engages with a component of an implantable device 1000, and then the driver shaft 164 is moved (e.g., axially) over the latch 122 and the component (e.g., a coupler) of the implantable device 1000 engaged with the latch 122 to hold such engaged components in place. In some embodiments, the driver 162 may be rotated to actuate the implantable device 1000 as desired, which also may result in rotation of the latch 122.

In accordance with various aspects of the present disclosure, a locking region 130 on the flexible elongate member 124 may be engaged by a portion of the driver shaft 164. For instance, the driver shaft 164 may include a locking region 170 configured to engage or correspond with a locking region 130 (either separate from locking regions 130 engaging with at least one of the latch 122 or control handle assembly 110 or latch knob 120, or an extension of one of such locking regions 130) on the flexible elongate member 124. Engagement of the locking region 130 on the flexible elongate member 124 with the locking region 170 of the driver shaft 164 may inhibit or prevent relative rotation between these components of the delivery/deployment system 100 while allowing axial advancement of the driver shaft 164 as desired to sheathe or unsheathe the engaged latch 122 and corresponding component of the implantable device 1000. Such engagement of the flexible elongate member 124 and driver shaft 164 may provide various benefits, such as reduction of torqueing of the flexible elongate member 124 with respect to the driver shaft 164. In addition, if the flexible elongate member 124 rotates along with the driver shaft 164 as a result of engagement of corresponding respective locking regions 130, 170, the torque on the driver shaft 164 (such as to actuate a component of the implantable device 1000) is enhanced or strengthened (and potential torsional fatigue reduced) in combination with the reinforcement by the flexible elongate member 124 resulting from interengagement of these components, resulting in a potentially more resilient or robust design than if the flexible elongate member 124 and the driver shaft 164 are not rotationally coupled to rotate together. In the example illustrated in FIG. 12, the driver shaft locking region 130 is in the form of a flat on each side of the flexible elongate member 124 and the driver shaft locking region 170 is in the form of corresponding flats within the channel 165 within the driver shaft 164 and through which the flexible elongate member 124 extends. In the example illustrated in FIG. 13, the driver shaft locking region 130 is in the form of a flat on one side of the flexible elongate member 124 and the driver shaft locking region 170 is in the form of a corresponding flat within the channel 165 within the driver shaft 164 and through which the flexible elongate member 124 extends.

As discussed above, a delivery/deployment system 100 formed in accordance with various principles of the present disclosure may be used to deliver and/or deploy and/or manipulate any of a variety of implantable devices. As also discussed above, the various control knobs 114 may be used to control various aspects of the implantable device via different motions of the associated components of the delivery/deployment system 100.

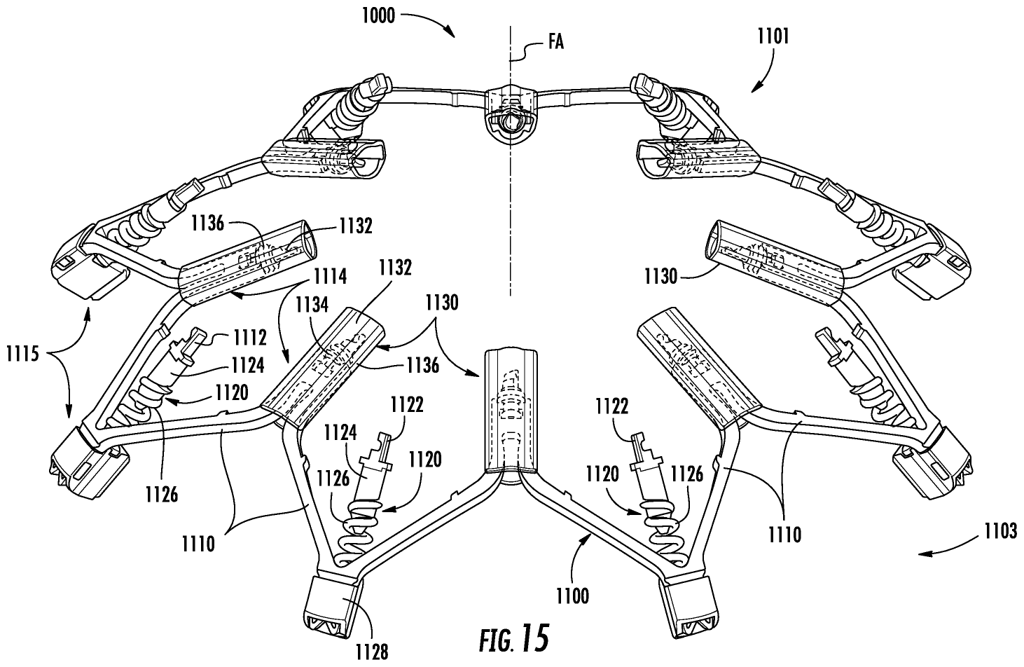

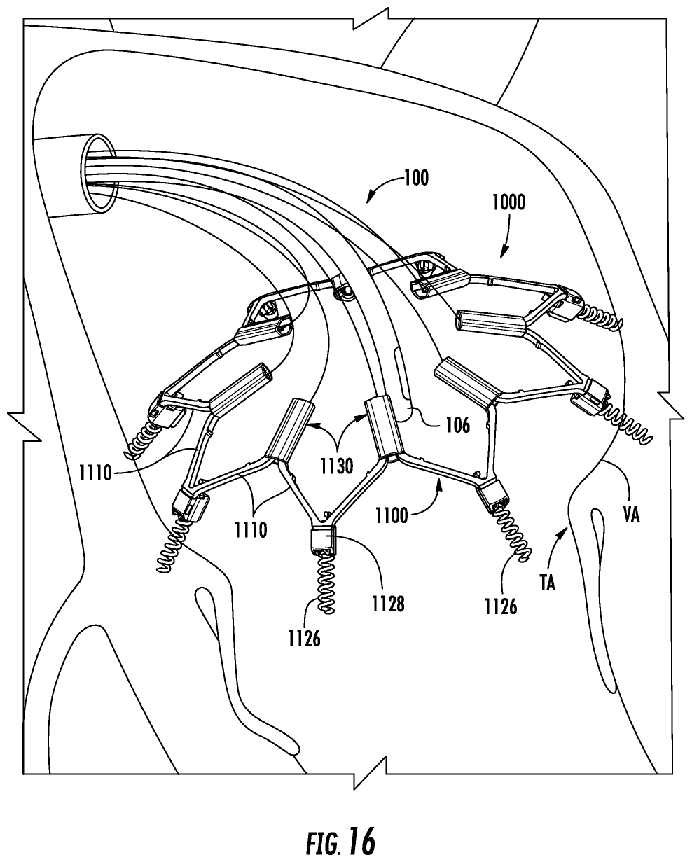

An example of a delivery/deployment system 100 formed in accordance with various principles of the present disclosure, and incorporating one or more aspects described herein, is shown in FIG. 1 delivering an example of an implantable device 1000 capable of moving between a compressed or retracted or collapsed delivery configuration (as illustrated in FIG. 1) and an expanded configuration (as illustrated in FIG. 14). The implantable device 1000 has a reduced diameter when in its compressed or retracted or collapsed (such terms being used interchangeably herein without intent to limit) configuration to facilitate delivery (such as through the delivery device 102, such as a delivery sheath or catheter, typically navigated through tortuous and/or narrow passages in the body, such as the vascular system) to the treatment site TS. An example of an implantable device 1000 is illustrated in further detail in FIG. 15 and FIG. 16. The implantable device 1000 in this example is an implantable device for annuloplasty, such as for custom reshaping of a heart valve (e.g., the mitral valve, as illustrated, or the tricuspid valve), and is capable of moving between the collapsed and expanded configurations and positions therebetween to modify the shape of the valve annulus VA at which it is implanted/to which it is secured. An imaging catheter 106 may be used to locate the treatment site TS at which the implantable device 1000 is to be delivered/deployed and implanted and/or to observe the configuration and/or position of the implantable device 1000 during implantation and adjustment. Once at the treatment site TS, the implantable device 1000, which may be held in a compressed or retracted or unexpanded (such terms being used interchangeably herein without intent to limit) configuration by a retention device or by the delivery device or otherwise, is allowed to expand for deployment and placement and implantation, as illustrated in FIG. 14. Expansion may occur naturally, for example if the frame is formed of a shape memory or super elastic material (e.g., Nitinol) that is biased towards an expanded state. In alternate embodiments, expansion may be mechanically controlled, for example through the use of a force applied within the frame using an expandable deployment device (e.g., an inflatable balloon or the like).

With reference to FIGS. 14-16, the illustrated example of an implantable device 1000 includes a frame member 1100 that may be disposed about a heart valve or other cardiac feature. The frame member 1100 may be generally symmetrical with respect to the central frame axis FA although it need not be symmetrical. The frame member 1100 may form a generally tubular shape, the term “tubular” being understood herein to include circular as well as other rounded or otherwise closed shapes. The frame member 1100 may be configured to change shape, size, dimension, and/or configuration. For example, the frame member 1100 may assume various shapes, sizes, dimensions, configurations etc. during different phases of deployment such as during pre-delivery, delivery, tissue engagement, anchoring, cinching, etc.

The frame member 1100 may be formed from one or more struts 1110 that may form all or part of the frame member 1100. The struts 1110 may include elongated structural members formed of a metal alloy, a shape memory material, such as an alloy of nickel titanium or other metals, metal alloys, plastics, polymers, composites, other suitable materials, or combinations thereof. In one embodiment, the struts 1110 may be formed from the same, monolithic piece of material (e.g., tube stock). Thus, reference to struts 1110 may refer to different portions of the same, extensive component. Alternatively, reference to struts 1110 may refer to components that are formed separately and attached together (optionally permanently, such as by welding or other methods). In some embodiments, the struts 1110 may be separate components that are detachably coupled to form proximal apices 1114 and distal apices 1115. Alternatively, if formed from a monolithic piece of material, the material may be cut or otherwise formed to define proximal apices 1114 and distal apices 1115. The frame member 1100 may be considered to be substantially tubular, and configured to change shape, size, dimensions, and/or configuration. For example, the frame member 1100 may assume various shapes, sizes, dimensions, configurations etc. during different phases of deployment such as during pre-delivery, delivery, tissue engagement, anchoring, and adjustment (e.g., cinching).

As shown in FIGS. 14-16, in the illustrated example of an embodiment of an implantable device 1000, a plurality of anchors 1120 are carried at a distal end 1103 of the frame member 1100, such as along the distal apices 1115 of the frame member 1100, and a plurality of collars or cinch collars or sleeves or cinch sleeves sliders or nuts 1130 (such terms being used interchangeably herein without intent to limit, reference being made generally to collars for the sake of convenience) are carried at the proximal apices 1114 of the frame member 1100. In the illustrated embodiments, the proximal end 1101 of the frame member 1100 is directed proximally toward and engaged or carried by the delivery/deployment system 100, and the distal end 1103 of the frame member 1100 extends distally from the delivery/deployment system 100 and is the end engaged with the treatment site TS. It will be appreciated that alternate configurations of the frame member 1100, such as depending on the manner and orientation in which the implantable device 1000 is delivered, are within the scope and spirit of the present disclosure.

As may be seen with reference to FIG. 1 and FIG. 14, one or more latches 122 of the delivery/deployment system 100 may engage a corresponding anchor latch 1122 on a respective anchor head 1124 (viewable in FIG. 15) on the frame member 1100, and one or more latches 122 may engage a corresponding collar actuator latch 1132 on a collar actuator head 1134 of a collar actuator 1136. The latches 1122 and 1132 on the frame member 1100, shown in further detail in FIG. 15, are configured to engage with the latches of the delivery/deployment system 100 to couple the associated components together such as for delivery of the implantable device 1000 to the treatment site TS. As may further be seen with reference to FIG. 1 and FIG. 14, one or more drivers 162 of the delivery/deployment system 100 may engage a respective anchor head 1122 (viewable in FIG. 15) on the frame member 1100, and one or more drivers 162 may engage a collar actuator head 1134 of the collar actuator 1136. The drivers 162 typically extend over the latches 122, 1122, 1132, such as to maintain engagement of the latches 122, 1122, 1132 with the implantable device 1000 (such as during delivery and/or manipulation of the frame member 1100 by the delivery/deployment system 100). Rotation of the drivers 162 coupled to the anchor heads 1124 causes advancement or withdrawal of the anchor shafts 1126 of the anchors 1120 with respect to the treatment site TS (in this example, a valve annulus) to implant, remove, or adjust the position of the frame member 1100. In some embodiments the anchors 1120 may translate through an anchor housing 1128 coupled to the frame member 1100. The anchor shaft 1126 (such as in the form of a helical shaft) may be coupled to and extend through a portion of an associated distal apex 1115, with or without an associated anchor housing 1128. Rotation of a driver 162 coupled to a collar actuator head 1134 causes advancement or withdrawal of the collar 1130 with respect to the proximal apex 1114 over which the collar 1130 is positioned to adjust the relative positions of the struts 1110 joined at such apex. Such adjustment results in adjustment of at least one of the size, shape, configuration, dimension, etc. of the frame member 1100 (e.g., retraction/compression or expansion of the frame upon bringing adjacent struts 1110 closer or further apart, respectively) to affect at least one of the size, shape, configuration, dimension, etc. of the treatment site TS (such as to restore or correct the shape of a valve annulus for proper functioning or competency thereof).

Various additional features of an implantable device 1000 as illustrated in FIGS. 14-16, as well as related delivery systems and methods of use may be appreciated with reference to the following patents and patent applications, each of which is incorporated herein by reference in its entirety for all purposes: U.S. Pat. No. 9,180,005 (Docket No. 8150.0563), issued Nov. 10, 2015, and titled “ADJUSTABLE ENDOLUMINAL MITRAL VALVE RING”; U.S. Pat. No. 10,335,275 (Docket No. 8150.0570), issued Jul. 2, 2019, and titled “METHODS FOR DELIVERY OF HEART VALVE DEVICES USING INTRAVASCULAR ULTRASOUND IMAGING”; U.S. Pat. No. 9,848,983 (Docket No. 8150.0568), issued Dec. 26, 2017, and titled “VALVE REPLACEMENT USING ROTATIONAL ANCHORS”; U.S. Pat. No. 10,555,813 (Docket No. 8150.0571), issued Feb. 11, 2020, and titled “IMPLANTABLE DEVICE AND DELIVERY SYSTEM FOR RESHAPING A HEART VALVE ANNULUS”; U.S. Pat. No. 10,548,731 (Docket No. 8150.0572), issued Feb. 4, 2020, and titled “IMPLANTABLE DEVICE AND DELIVERY SYSTEM FOR RESHAPING A HEART VALVE ANNULUS”; U.S. Pat. No. 9,192,471 (Docket No. 8150.0564), issued Nov. 24, 2015, and titled “DEVICE FOR TRANSLUMINAL RESHAPING OF A MITRAL VALVE ANNULUS”; U.S. Patent Application Publication No. 2010/0249920 (Docket No. 8150.0564X), published Sep. 30, 2010, and titled “DEVICE FOR TRANSLUMINAL RESHAPING OF A MITRAL VALVE ANNULUS”; U.S. Pat. No. 9,795,480 (Docket No. 8150.0565D), issued Oct. 24, 2017, and titled “RECONFIGURING TISSUE FEATURES OF A HEART ANNULUS”; U.S. Pat. No. 9,610,156 (Docket No. 8150.0566), issued Apr. 4, 2017, and titled “MITRAL VALVE INVERSION PROSTHESES”; and/or U.S. Pat. No. 10,321,999 (Docket No. 8150.0569), issued Jun. 18, 2019, and titled “SYSTEMS AND METHODS FOR RESHAPING A HEART VALVE”. Thus, the description of particular features and functionalities herein is not meant to exclude other features and functionalities, such as those described in the references incorporated herein by reference or others within the scope of the development.

Although embodiments of the present disclosure may be described with specific reference to an implant for use with mitral valves or tricuspid valves, it is appreciated that various other implants may similarly benefit from the structures and manufacturing methods disclosed herein. For example, implants which must withstand the palpatory forces for repairing a tricuspid valve annulus and/or addressing other dilatation, valve incompetency, valve leakage and other similar heart failure conditions may also benefit from the concepts disclosed herein. Principles of the present disclosure may be applied to other delivery/deployment devices subject to torsional forces. Principles of the present disclosure may be applied to other delivery/deployment systems and/or implants, such as those disclosed in U.S. Pat. No. 10,575,853, issued Mar. 3, 2020, and titled EMBOLIC COIL DELIVERY AND RETRIEVAL; U.S. Pat. No. 10,548,605, issued Feb. 4, 2020, and titled DETACHABLE IMPLANTABLE Devices; and U.S. Pat. No. 10,478,192, issued Nov. 19, 2019, and titled DETACHABLE MECHANISM FOR IMPLANTABLE DEVICES, each of which patents is incorporated by reference herein in its entirety for all purposes.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The following claims are hereby incorporated into this Detailed Description by this reference, with each claim standing on its own as a separate embodiment of the present disclosure. Reference signs in the claims are provided merely as a clarifying example and shall not be construed as limiting the scope of the claims in any way.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “the”, “first”, “second”, etc., do not preclude a plurality. Reference signs in the claims are provided merely as a clarifying example and shall not be construed as limiting the scope of the claims in any way.

What is claimed is:

1. A delivery and deployment system for delivering and/or deploying an implantable device, the system comprising:
a control handle configured to manipulate components of the delivery and deployment device;
a flexible elongate member having a proximal end coupled to the control handle and a distal end extending distally therefrom; and
a latch coupled to the distal end of the flexible elongate member;
wherein:
the flexible elongate member comprises a first locking region, formed on an exterior thereof, shaped and configured to engage with one of the control handle or the latch to inhibit relative movement therebetween; and
the one of the control handle or the latch comprises a channel for receiving an end of the flexible elongate member with a channel locking region corresponding to the first locking region for engagement therewith to inhibit relative movement between the flexible elongate member and the one of the control handle and the latch.

2. The system of claim 1, wherein the first locking region has a shape and/or configuration distinct from the contour of the flexible elongate member surrounding the first locking region.

3. The system of claim 2, wherein the first locking region is at least one of a flat, a swaged region, or an enlarged region.

4. The system of claim 1, further comprising at least one latch control knob associated with the control handle, the proximal end of the flexible elongate member engaging the control handle via the latch control knob, wherein the latch control knob defines a channel for receiving the proximal end of the flexible elongate member and having a locking region corresponding to and engaging with the first locking region to inhibit relative movement between the flexible elongate member and the latch control knob.

5. The system of claim 1, wherein the first locking region is a flat on the exterior of the flexible elongate member, and the control handle includes a press-fit locking element extending through a portion of the control handle and into engagement with the flat.

6. The system of claim 1, further comprising a driver shaft defining a channel therethrough, the flexible elongate member extending through the channel in the driver shaft.

7. The system of claim 6, wherein the driver shaft channel comprises a driver shaft channel locking region, and the flexible elongate member comprises a second locking region formed in an exterior thereof shaped and configured to engage with the driver shaft channel locking region to inhibit relative rotation, while allowing axial movement, between the flexible elongate member and the driver shaft.

8. The system of claim 7, wherein:
the control handle includes at least one actuator control knob; and
the driver shaft has a proximal end coupled to the actuator control knob and a distal end coupled to a driver coupler configured to engage a component of an implantable device, movement of the actuator control knob controlling movement of the driver coupler via the driver shaft.

9. The system of claim 8, wherein the control handle further includes at least one latch control knob located proximal to the actuator control knob, the flexible elongate member extending through the actuator control knob and into the latch control knob.

10. The system of claim 7, wherein the first locking region and the second locking region are separate.

11. The system of claim 7, wherein the second locking region is an extension of the first locking region.

12. An implantable device delivery/deployment system comprising:
a flexible elongate member having a proximal end coupled to the control handle and a distal end extending distally therefrom;
a latch coupled to the distal end of the flexible elongate member;
an implantable device having a coupler corresponding to and configured to be engaged with the latch; and
a control handle configured to manipulate components of the delivery/deployment system to deliver and deploy the implantable device;
wherein the flexible elongate member comprises a first locking region formed in an exterior thereof shaped and configured to engage with one of the control handle or the latch to inhibit relative movement therebetween.

13. The system of claim 12, wherein the one of the control handle or the latch comprises a channel for receiving an end of the flexible elongate member with a channel locking region corresponding to the first locking region for engagement therewith to inhibit relative movement between the flexible elongate member and the one of the control handle and the latch.

14. The system of claim 12, further comprising a driver shaft defining a channel therethrough and having a proximal end coupled to the control handle and a distal end extending distally therefrom configured to engage a component of the implantable device to deploy the implantable device at a treatment site, wherein:
the flexible elongate member extends through the channel defined through the driver shaft;
the driver shaft comprises a driver shaft channel locking region; and
the flexible elongate member comprises a second locking region formed in an exterior thereof shaped and configured to engage with the driver shaft channel locking region.

15. The system of claim 14, further comprising:
a latch control knob coupled to the proximal end of the flexible elongate member;
a driver coupler coupled to the distal end of the driver shaft and configured to extend over the latch and corresponding coupler on the implantable device; and
an actuator control knob coupled to the proximal end of the driver shaft;
wherein:
actuation of the actuator control knob actuates the driver shaft to rotate to manipulate a portion of the implantable device, or to move axially relative to the latch to sheathe or unsheathe the latch and the corresponding coupler on the implantable device; and
rotational movement between the flexible elongate member and the driver shaft is inhibited by engagement of the second locking region and the driver shaft channel locking region.

16. A method of delivering and deploying an implantable device to a treatment site, the method comprising:
coupling a latch at a distal end of a flexible elongate member extending from a delivery/deployment system to a coupler on the implantable device;
sheathing the coupled latch and coupler with a driver shaft extending from the delivery/deployment system to maintain engagement between the latch and the coupler during delivery of the implantable device to the treatment site, wherein the flexible elongate member extends through a channel defined in the driver shaft and has a locking region on an exterior thereof engaged with a corresponding locking region in the driver shaft channel to inhibit relative rotational movement between the flexible elongate member and the driver shaft; and
navigating the flexible elongate member and driver shaft to deliver the implantable device to the treatment site.

17. The method of claim 16, further comprising actuating the driver shaft via an actuator control knob on a control handle of the delivery/deployment system to implant an anchor of the implantable device into the treatment site.

18. The method of claim 16, further comprising actuating the driver shaft via an actuator control knob to adjust at least one of the size, shape, configuration, or dimension of the implantable device.

19. The method of claim 16, further comprising extending a driver coupler on a distal end of the driver shaft over the latch and engaging the driver coupler with the coupler on the implantable device, and rotating the driver shaft via an actuator control knob to rotate a component of the implantable device coupled to rotate with the coupler to adjust at least one of the size, shape, configuration, dimension, or position of the implantable device, the flexible elongate member engaging within and reinforcing rotation of the driver shaft.

20. The method of claim 16, further comprising moving the driver shaft proximally with respect to the flexible elongate member to allow the latch to disengage from the corresponding coupler on the implantable device.
